(19) United States
(12) Patent Application Publication
(10) Pub. No.: US 2026/0224381 A1
(43) Pub. Date: Aug. 6, 2026

(54) AI-Integrated External Gastric Stimulation System for Appetite Control and Behavioral Modification

(71) Applicant: Appetec, Inc., Macon, GA (US)

(72) Inventor: Shahriar Sedghi, Macon, GA (US)

(73) Assignee: Appetec, Inc., Macon, GA (US)

(21) Appl. No.: 19/043,337

(22) Filed: Jan. 31, 2025

(51) Int. Cl. A61F 5/00; A61B 5/00; G16H 20/30; G16H 40/63; G16H 80/00

(52) U.S. Cl. CPC A61F 5/0003; A61B 5/0002; A61B 5/4833; A61B 5/4836; A61B 5/7475; G16H 20/30; G16H 40/63; G16H 80/00

(57) ABSTRACT

An external gastric vibrational device for inducing satiety and supporting weight management comprises an external vibrational device configured to transmit intragastric soundwaves to stimulate a stomach non-invasively. The device features adjustable frequency and intensity settings and includes multi-layer acoustic insulation and vibration-dampening structures to minimize operational noise. A mobile device communicatively connected to the external vibrational device comprises an artificial intelligence (AI)-driven mobile application configured to determine optimal therapy timing based on physiological and behavioral data, prompt the user accordingly, continuously learn from user responses to adjust therapy parameters, track trends in weight and dietary habits to provide personalized feedback, and provide a virtual reality (VR) module to deliver calming or aversive virtual environments corresponding to user mood states to address emotional eating and support behavioral modification.

### SUMMARY

The present patent disclosure introduces an external vibrational device (with intragastric soundwave transmission) designed to induce a feeling of satiety by stimulating the stomach non-invasively. The device aims to provide a minimally invasive weight loss solution for both morbidly obese patients and those who are moderately overweight, offering a safer alternative to traditional surgical procedures.

The vibrational device operates by activating vagal nerve fibers through external gastric stimulation. It features customizable frequency and intensity settings, allowing personalized therapy based on individual physiological needs. The device is designed to be adaptable in handheld and wearable formats, enhancing user convenience and compliance.

Additional enhancements include soundproofing solutions such as acoustic insulation materials, frequency optimization algorithms, and adaptive noise-canceling technology, ensuring silent operation for user comfort.

Customizable therapy schedules are dynamically adjusted using AI analytics that factor in metabolic responses, meal patterns, and behavioral triggers A key innovation of the invention is its bidirectional communication with a companion app, which facilitates real-time tracking, compliance monitoring, and therapy optimization. The device builds on existing FDA-approved models while integrating advanced features and AI capabilities for improved effectiveness and user engagement.

Although specific advantages have been enumerated above, various embodiments may include some, none, or all of the enumerated advantages.

Additionally, other technical advantages may become readily apparent to one of ordinary skill in the art after review of the following figures and description.

### FIGURES

FIG. 1: Interface between Vibration Device and Mobile App

FIG. 2: Workflow for Promoting Effective Behavior Modification

### DETAILED DESCRIPTION

The proposed external intragastric vibrational device is an advanced, non-invasive tool designed to activate vagal nerve fibers and promote satiety by stimulating the stomach externally. This device leverages customizable frequency and intensity settings, enabling it to cater to the unique physiological needs of each user. The system's versatile design supports both handheld and wearable formats, ensuring convenience and flexibility in various lifestyle scenarios.

A key innovation of the device is its bidirectional communication with an AI-driven mobile application, allowing for real-time tracking, compliance monitoring, and therapy optimization. By building upon existing FDA-approved models, the device incorporates cutting-edge features that enhance its therapeutic effectiveness and enable seamless AI integration.

The application will introduce virtual reality to combat mood eating disorders, specifically designed for patients with such disorders. Patients will be prompted to participate in motivational challenges to encourage device use and diet/exercise compliance.

The device integrates advanced soundproofing mechanisms, such as multi-layer acoustic insulation and vibration dampening structures, to minimize operational noise and ensure discretion. The addition of a silent mode feature within the mobile application enables users to activate low-noise operation when needed.

The application introduces virtual reality (VR) to combat mood-related eating disorders, specifically targeting patients with emotional eating behaviors. Patients will engage in personalized VR experiences designed to enhance satiety cues and improve adherence to dietary guidelines.

The device functions in conjunction with an AI-powered mobile application, which forms the cornerstone of the overall weight management method. The application prompts users to utilize the vibrational device based on AI-driven analytics that determine the optimal time and duration of therapy. This determination is based on a wide array of physiological and behavioral data collected through the app, considering critical factors such as:
Eating habits
Physical activity
Sleep patterns
Stress levels
Hormonal fluctuations

The AI-enhanced mobile app integrates diverse physiological and behavioral inputs, such as glucose levels, eating habits, physical activity, and sleep patterns. By continuously learning from user data, the app provides personalized recommendations for vibration therapy and lifestyle modifications. It tracks trends in weight, dietary habits, and activity while offering real-time feedback and educational content tailored to user queries. Compatibility with CGMs and smartwatches enhances its data-driven approach, creating a comprehensive and adaptive weight loss solution.

The app's VR modules address emotional eating by targeting specific triggers. Using a mood tracker, the app identifies correlations between emotional states and eating behaviors. For instance, users experiencing sadness and sugar cravings may be guided to calming VR scenarios, such as swimming with dolphins, paired with protein-rich meals to promote satiety and positive emotions. Conversely, aversive VR therapy reconditions responses to unhealthy foods by pairing gastric stimulation with unpleasant VR scenarios, discouraging addictive eating habits. These interventions are evidence-based and designed to support emotional regulation and sustainable behavior changes.

The proposed approach offers several distinct advantages over current obesity treatment modalities. The method prioritizes patient safety and avoids invasive procedures, enhancing its accessibility and acceptance. Obesity is a multifactorial condition requiring tailored interventions. The AI-enhanced mobile application leverages personalized behavioral insights and AI capabilities to optimize the timing and efficacy of gastric vibrational therapy, resulting in improved weight loss outcomes.

By integrating with smartphones, the approach ensures seamless data transmission and user engagement, aligning with daily habits for minimal disruption to routine. The app will include a chatbot feature, enabling users to seek guidance and/or clarification on optimal weight loss strategies, thus fostering sustained adherence.

Recognizing that long-term behavior modification is essential for addressing obesity, this technology provides continuous support that adapts to the individual and reinforces lifelong healthy habits.

This approach can be applied to and benefit younger adults, individuals with a BMI under 30, as well as those with a history of obesity (increased lifelong risk). This expands the intervention's relevance and potential impact.

This innovative integration of AI, personalized care, and patient-centric design positions the proposed method as a transformative solution for obesity management.

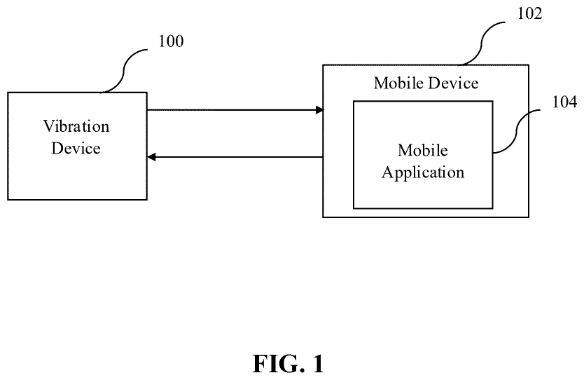

FIG. 1 illustrates the interface between the external vibrational device and the AI-driven mobile application. The device communicates wirelessly with the app, enabling seamless data transfer and real-time interaction. The interface is designed to promote user engagement by providing real-time feedback on therapy sessions, monitoring compliance, and offering personalized recommendations. The synchronization between the device and the app ensures that users receive optimal therapy tailored to their individual needs, enhancing the overall effectiveness of the weight management program.

To enhance personalization, the device integrates with health monitoring systems such as CGMs and smartwatches, offering seamless data collection for a comprehensive weight management solution. The device dynamically adjusts its vibration patterns based on real-time user data, improving therapy efficacy.

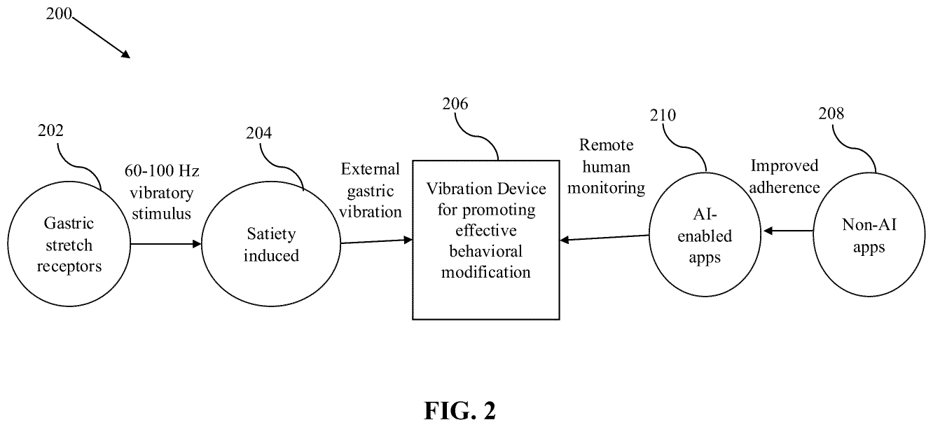

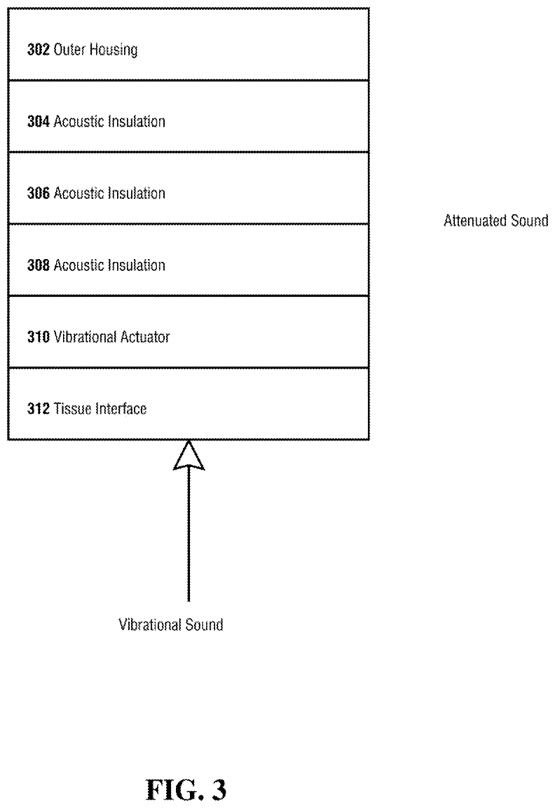

FIG. 2 depicts the workflow for promoting effective behavior modification utilizing satiety induced by gastric stimulation and AI-enabled applications. The process begins with the AI-driven analytics, which assess the user's physiological and behavioral data to determine the optimal timing and settings for the vibrational device. The mobile application then prompts the user to initiate the therapy session, providing motivational challenges and tracking progress. The integration of virtual reality experiences aims to combat mood-related eating disorders by reinforcing positive dietary behaviors and enhancing satiety cues. The continuous feedback loop between the device, app, and user fosters long-term adherence to the weight management program, ensuring sustainable results.

The system includes an option for remote human oversight, enabling healthcare providers to monitor therapy compliance, review data trends, and offer personalized recommendations via the mobile application interface. This feature ensures professional guidance in conjunction with AI-driven recommendations, allowing for adjustments to therapy protocols based on clinical assessments. The integration of human oversight enhances patient confidence, fosters adherence, and ensures safe, effective use of the device

The device features user-configurable settings accessible via the mobile application or directly on the device, allowing users to manually adjust frequency and intensity based on personal comfort and preference. AI algorithms dynamically adapt these settings based on real-time physiological and behavioral data, optimizing therapy efficacy. This dual control mechanism ensures a personalized experience while leveraging automated intelligence to enhance treatment. The vibration device can be turned on or adjusted manually, through AI/app notification or directly by AI.

1. (canceled)

2. (canceled)

3. (canceled)

4. (canceled)

5. (canceled)

6. An external gastric vibrational device for inducing satiety and supporting weight management in a user, said external gastric vibrational device comprising:
an external vibrational device configured to transmit gastric soundwaves to induce a feeling of satiety by stimulating a stomach of said user non-invasively, wherein a frequency and intensity of said external vibrational device are adjustable, and wherein said external vibrational device comprises multi-layer acoustic insulation configured to minimize operational noise and ensure discreet operation; and
a mobile device communicatively connected to said external vibrational device, wherein said mobile device comprises an artificial intelligence (AI)-driven mobile application, wherein said AI-driven mobile application is configured to:
determine an optimal time and duration of therapy based on physiological and behavioral data, said physiological and behavioral data comprising eating habits, physical activity, sleep patterns, stress levels, hormonal fluctuations, and glucose levels;
prompt said user to utilize said external vibrational device based on said optimal time and duration;
autonomously activate said external vibrational device without user input when said determination of said physiological and behavioral data indicates therapeutic intervention is optimal, wherein said autonomous activation occurs directly through artificial intelligence control without requiring user confirmation, and wherein said AI-driven mobile application identifies, from said physiological and behavioral data, patterns in said eating habits, said physical activity, said sleep patterns, said stress levels, said hormonal fluctuations, and said glucose levels that are predictive of an impending eating episode, and determines said optimal time and duration of therapy based on said patterns independently of hunger detection by said user;
continuously learn from user responses and data to dynamically adjust parameters of said therapy to improve personalization for said user;
track and analyze trends in weight, dietary habits, and activity to provide personalized feedback, educational guidance, and lifestyle modification recommendations; and
provide a virtual reality (VR) module configured to deliver calming or aversive virtual environments corresponding to user mood states to address emotional eating and support behavioral modification, wherein said VR module uses a mood tracker to identify correlations between emotional states and eating behaviors, and delivers said calming virtual environments when said user experiences negative emotional states associated with sugar cravings and delivers said aversive virtual environments to recondition responses to unhealthy foods, said mood tracker identifying said emotional states based at least in part on said stress levels and said hormonal fluctuations from said physiological and behavioral data.

7. The external gastric vibrational device of claim 6, wherein said external gastric vibrational device is configured to interface with continuous glucose monitors (CGMs) and smartwatches to enhance said therapy.

8. The external gastric vibrational device of claim 6, wherein said AI-driven mobile application comprises a mood tracker to identify correlations between said mood states and eating behaviors.

9. The external gastric vibrational device of claim 6, wherein said external gastric vibrational device is adaptable in a handheld or a wearable configuration.

10. A method for inducing satiety and supporting weight management in a user, said method comprising the steps of:
transmitting gastric soundwaves to induce a feeling of satiety by stimulating a stomach of said user non-invasively using an external vibrational device, a frequency and intensity of said external vibrational device being adjustable, and said external vibrational device comprising multi-layer acoustic insulation configured to minimize operational noise and ensure discreet operation;
determining an optimal time and duration of therapy based on physiological and behavioral data, eating habits, physical activity, sleep patterns, stress levels, hormonal fluctuations, and glucose levels, using an artificial intelligence (AI)-driven mobile application executing on a mobile device communicatively connected to said external vibrational device;
prompting said user to utilize said external vibrational device based on said optimal time and duration;
autonomously activating said external vibrational device without user input when said determination of said physiological and behavioral data indicates therapeutic intervention is optimal, said autonomous activation occurring directly through artificial intelligence control without requiring user confirmation; wherein said AI-driven mobile application identifies. from said physiological and behavioral data, patterns in said eating habits, said physical activity, said sleep patterns, said stress levels, said hormonal fluctuations, and said glucose levels that are predictive of an impending eating episode, and determines said optimal time and duration of therapy based on said patterns independently of hunger detection by said user;
continuously learning from user responses and data to dynamically adjust parameters of said therapy to improve personalization for said user;
tracking and analyzing trends in weight, dietary habits, and activity to provide personalized feedback, educational guidance, and lifestyle modification recommendations; and
delivering calming or aversive virtual environments corresponding to user mood states to address emotional eating and support behavioral modification using a virtual reality (VR) module of said AI-driven mobile application, wherein said VR module uses a mood tracker to identify correlations between emotional states and eating behaviors, and delivers said calming virtual environments when said user experiences negative emotional states associated with sugar cravings and delivers said aversive virtual environments to recondition responses to unhealthy foods, said mood tracker identifying said emotional states based at least in part on said stress levels and said hormonal fluctuations from said physiological and behavioral data.

11. The method of claim 10, further comprising interfacing said external gastric vibrational device with continuous glucose monitors (CGMs) and smartwatches to enhance said therapy.

12. The method of claim 10, further comprising providing access to remote devices operated by healthcare providers for providing real-time feedback on said therapy, monitoring compliance, and offering personalized recommendations.
